(19) United States
(12) Patent Application Publication
(10) Pub. No.: US 2018/0215697 A1
(43) Pub. Date: Aug. 2, 2018

(54) BIS(OXALATO) PLATINUM ACID "ON THE ROCKS"

(71) Applicant: UMICORE AG & CO. KG, Hanau-Wolfgang (DE)

(72) Inventors: Eileen WOERNER, Nidderau (DE); Ralf KARCH, Kleinostheim (DE); Andreas RIVAS-NASS, Bensheim (DE); Angelino DOPPIU, Seligenstadt (DE); Annika FREY, Hanau (DE)

(73) Assignee: UMICORE AG & CO. KG, Hanau-Wolfgang (DE)

(21) Appl. No.: 15/747,480

(22) PCT Filed: Jul. 27, 2016
(86) PCT No.: PCT/EP2016/067944

§ 371 Date: Jan. 25, 2018

(30) Foreign Application Priority Data
Jul. 28, 2015 (EP) 15178718.1

(51) Int. Cl. C07C 55/07; C07C 51/41; B01J 2/18; B01J 2/06

(52) U.S. Cl. CPC C07C 55/07; B01J 2/06; B01J 2/18; C07C 51/418

(57) ABSTRACT

The present invention relates to a method for the stabilization of bis(oxalato) platinum (II) acid and to a device therefor.

Bis(oxalato) platinum (II) acid is a compound known in the literature, and is described, for instance, in Krogmann, K., Dodel P., Chem. Ber. 99 (1966) 3408-3418.

This compound may be used, for instance, in the synthesis of catalysts, as described in WO 2013/103396 and WO 2013/130796.

One advantage of this compound over other platinum sources lies in the fact that it contains neither metal ions (such as alkali metals or alkaline earth metals) nor halogens.

While emitting carbon dioxide, the compound releases platinum, so that no undesired residues can contaminate the desired product.

What is disadvantageous, however, is the instability of aqueous solutions of bis(oxalato) platinum (II) acid (surprisingly, not described in the literature), since this reaction takes place spontaneously even at room temperature and leads to decomposition, so that, after some time, there is no longer any bis(oxalato) platinum (H) acid present.

Since bis(oxalato) platinum (II) acid forms a blue solution and, at greater dilutions, a yellow solution, but, after decomposition, there remains a light brownish suspension of platinum in water—possibly, with the deposition of a platinum mirror—this decomposition reaction is easily observed.

### OBJECT OF THE INVENTION

The need arose, therefore, to find a simple means suitable for improving the storage stability of aqueous solutions of bis(oxalato) platinum (II) acid over a longer period of time. This aim is achieved by the freezing of the aqueous solution of bis(oxalato) platinum (II) acid.

### BRIEF DESCRIPTION

1. Method for improving the storage stability of aqueous solutions of bis(oxalato) platinum (II) acid, comprising the steps of (1) preparing an aqueous solution of bis(oxalato) platinum (II) acid; (2) cooling the aqueous solution of bis(oxalato) platinum (II) acid to a temperature below its freezing point until complete solidification.

2. Method according to point 1, wherein the preparation of the aqueous solution of bis(oxalato) platinum (II) acid is accomplished by dissolving bis(oxalato) platinum (II) acid in water or by the production of bis(oxalato) platinum (II) acid as an aqueous solution.

3. Method according to point 1 or 2, wherein the preparation of the aqueous solution of bis(oxalato) platinum (II) acid is accomplished by the reaction of a precious metal precursor with oxalic acid, one of its hydrates or salts in water.

4. Method according to point 3, wherein the precious metal precursor is selected from the group consisting of hexahydroxo platinum (IV) acid, its salts, precious metal oxide hydrate, precious metal nitrate, precious metal acetate, and mixtures thereof.

5. Method according to one or more of points 2 through 4, wherein, to produce bis(oxalato) platinum (II) acid as an aqueous solution, bis(oxalato) platinum (II) acid is added to the reaction mix.

6. Method according to one or more of points 2 through 5, wherein the production of bis(oxalato) platinum (II) acid as an aqueous solution is accomplished at temperatures of 0° C. to 20° C., 5° C. to 60° C., 30° C. to 56° C., or 35° C. to 52° C., or at 45° C. to 55° C.

7. Method according to one or more of points 2 through 6, wherein, to produce bis(oxalato) platinum (II) acid as an aqueous solution, in a first step, an aqueous solution or suspension containing at least precious metal precursor and possibly bis(oxalato) platinum (II) acid is produced,
in a second step, the aqueous solution or suspension from the first step is heated to reaction temperature, and, in a third step, oxalic acid, one of its hydrates, or oxalic acid salt is added.

8. Method according to one or more of points 2 through 7, wherein, to produce bis(oxalato) platinum (II) acid as an aqueous solution, oxalic acid, oxalic acid dihydrate, sodium oxalate, ammonium oxalate, potassium oxalate, or mixtures thereof are used.

9. Method according to one or more of points 3 through 8, wherein the oxalic acid or one of its hydrates or oxalic acid salt is added in an amount of 1.7 to 3.1 molar equivalents, 1.8 to 2.8 molar equivalents, or 2 to 3 molar equivalents, relative to the platinum in the precious metal precursor.

10. Method according to one or more of points 3 through 9, wherein, in a first step, a suspension of platinum(IV) hydroxo acid in water is prepared; if necessary, in a second step, a solution of bis(oxalato) platinum (II) acid in water is produced; if necessary, the suspensions from the first and second steps are combined, and the resulting mixture is heated to the reaction temperature; in a third step, a first portion of 0.5 to 1.6—particularly, 0.4 to 1.4—molar equivalents of oxalic acid relative to platinum in platinum(IV) hydroxo acid is added; and, in a fourth step, a second portion of 0.3 to 1.5 or 0.1 to 1.4 molar equivalents of oxalic acid relative to platinum in platinum(IV) hydroxo acid is added; and, in a fifth step, step four is optionally repeated one to several times until a total amount of 1.7 to 3.1, or 1.8 to 2.8, or 2 to 3 molar equivalents of oxalic acid relative to platinum in (platinum(IV) hydroxo acid) is added.

11. Shaped object of ice, containing bis(oxalato) platinum (II) acid.

12. Shaped object according to point 11, which has an approximately spherical, cuboid, cylindrical, conical, truncated pyramidal, angular, or prismatic shape, or combinations thereof.

13. Shaped object according to point 11 or 12, which contains 1 wt % to 40 wt %—particularly, 19 wt % to 27 wt %—of bis(oxalato) platinum (II) acid.

14. Method for producing the shaped object according to one or more of points 11 through 13, wherein an aqueous solution of bis(oxalato) platinum (II) acid is poured into a mold and cooled to a temperature below its freezing point until complete solidification.

15. Method for producing the shaped object according to one or more of points 11 through 13, wherein, to produce the approximately spherical particles, an aqueous solution of bis(oxalato) platinum (II) acid is guided through at least one opening to form droplets, wherein, to form the approximately spherical particles, the droplets at the exit of the opening are guided to a cooling medium for solidification, the temperature of which is below the freezing point of the aqueous solution of bis(oxalato) platinum (II) acid.

16. Method according to point 15, wherein the opening is a nozzle.

17. Method according to point 15 or 16, wherein the jet of liquid exiting the opening is exposed to vibrations.

18. Method according to point 16, wherein the opening or nozzle is immersed in the cooling medium.

19. Method according to one or more of points 15 through 18, wherein the temperature of the cooling medium continuously decreases with increasing distance from the opening or nozzle (7).

20. Method according to one or more of points 15 through 19, wherein the temperature of the cooling medium in the region of the opening or nozzle is at least 10° C. below the freezing point of the aqueous solution of bis(oxalato) platinum (II) acid.

21. Method according to one or more of points 15 through 20, wherein liquid cooling medium, gaseous cooling medium, or a combination thereof is used.

22. Method according to one or more of points 15 through 21, wherein nitrogen or carbon dioxide is used as the cooling medium.

23. Method according to one or more of points 15 through 22, wherein the liquid jet exiting the opening is exposed to vibrations of constant frequency and amplitude.

24. Device for conducting the method according one of points 15 through 23, with means for producing droplets from an aqueous solution of bis(oxalato) platinum (II) acid, which comprises means for producing vibrations within a liquid jet and at least one opening—particularly, a nozzle—through which the liquid jet is guided, wherein, to produce spherical particles of an aqueous solution of bis(oxalato) platinum (II) acid, the droplets at the exit of the opening or nozzle are guided to a cooling medium, wherein the opening or nozzle (7) is possibly immersed in the cooling medium (10), and the temperature of the cooling medium (10) is below the freezing point of the aqueous solution of bis(oxalato) platinum (II) acid (3).

25. Device according to point 24, wherein the aqueous solution of bis(oxalato) platinum (II) acid (3) is disposed in a vessel (4), in which a vibrating plate is introduced as a means for producing vibrations.

26. Device according to point 24 or 25, wherein the underside of the vessel (4) terminates in a tubular outlet (6), via which the aqueous solution of bis(oxalato) platinum (II) acid (3) is guided to the opening or nozzle (7).

27. Device according to one of points 24 through 26, wherein a vibrating element (9) is coupled to the opening or nozzle (7) and/or to the outlet (6), by means of which the opening or nozzle (7) is vibrated.

28. Device according to one of points 24 through 27, wherein the vibrating element (9) is formed from a piezoelectric element or an electromagnetic vibrating element (9).

29. Device according to one of points 24 through 28, wherein the inner diameter of the opening or nozzle and/or the frequency of the vibrations is adjusted to the dimensions of the particles (2) to be produced.

30. Device according to one of points 24 through 29, wherein this has a multiple arrangement of openings or nozzles (7).

31. Device according to one of points 24 through 30, wherein this has means for the continuous supply of the aqueous solution of bis(oxalato) platinum (II) acid (3).

32. Device according to one of points 24 through 31, wherein the cooling medium (10) is disposed in a collection vessel (11) having a hollow, cylindrical column (13), wherein the opening or nozzle (7) is guided via the open upper end of the column (13) into the collection vessel (11).

33. Device according to one of points 24 through 32, wherein the temperature of the cooling medium (10) in the column (13) continuously decreases from the upper side to the lower end of the column (13).

34. Device according to one of points 24 through 33, wherein, in the region of the bottom of the collection vessel (11), a discharge device (14) for the removal of particles (2) from the collection vessel (11) is provided.

35. Device according to one of points 24 through 34, wherein the opening or nozzle (7) and the outlet (6) may be heated by means of a heating element.

36. Method for producing the shaped object according to one or more of points 11 through 13, wherein the aqueous solution of bis(oxalato) platinum (II) acid is guided via at least one opening to a cooling medium, the temperature of which is below the freezing point of the aqueous solution of bis(oxalato) platinum (II) acid and which is stirred in order to produce particles.

37. Method according to point 36, wherein the cooling medium is located in a vessel.

38. Method according to point 36 or 37, wherein the cooling medium is stirred by a mixer having a movable container, such as a pan mixer, planetary mixer or ring-trough mixer, or mixers having movable mixing tools such as a propeller stirrer, impeller stirrer, swash-plate stirrer, hollow-blade stirrer, crossbeam stirrer, anchor stirrer, paddle stirrer, grid stirrer, spiral stirrer, residual quantity stirrer, or combinations thereof.

39. Method according to one or more of points 36 or 37, wherein the cooling medium is a liquefied gas—particularly, liquefied nitrogen.

40. Method according to one or more of points 1 through 10 or shaped object according to one or more of points 11 through 13, wherein, after 35 days—particularly, after 78 days or 101 days—the aqueous solutions of bis(oxalato) platinum (II) acid demonstrate a loss in weight of less than 2 wt %, less than 1 wt % or 0.5 wt %—particularly, no loss in weight.

41. Method for producing the shaped object according to one or more of points 11 through 13, comprising the steps of (1) providing an aqueous solution of bis(oxalato) platinum (II) acid; (2) cooling the aqueous solution of bis(oxalato) platinum (II) acid to a temperature below its freezing point until complete solidification; (3) grinding the frozen aqueous solution of bis(oxalato) platinum (II) acid obtained in the previous step.

42. Method according to point 41, wherein the preparation—step (1)—is performed according to one or more of points 3 through 9.

### DETAILED DESCRIPTION

In a first step, an aqueous solution of bis(oxalato) platinum (II) acid is prepared, and, in a subsequent step, this is cooled to a temperature below its freezing point until complete solidification.

The aqueous solution of bis(oxalato) platinum (II) acid may contain additional components or auxiliary substances such as, for instance, oxalic acid or elemental platinum, but also thickeners or thixotropic agents, which either serve to produce the frozen particles from the aqueous solution of bis(oxalato) platinum (II) acid in the desired form or make this easier, or else are necessary for the subsequent application and would otherwise have to be added separately.

However, it must be noted that these additives must not be detrimental to the stability of the bis(oxalato) platinum (II) acid.

In one embodiment, the aqueous solution of bis(oxalato) platinum (II) acid substantially consists of water, bis(oxalato) platinum (II) acid, and unavoidable contaminants. Unavoidable contaminants include platinum, oxalic acid, and hexahydroxo platinum (IV) acid.

The production of the aqueous solution of bis(oxalato) platinum (II) acid is accomplished by dissolving bis(oxalato) platinum (II) acid in water or by the direct production of bis(oxalato) platinum (II) acid as an aqueous solution. Since pure bis(oxalato) platinum (II) acid as a solid can decompose in an exothermic reaction, it is almost always produced and stored in the form of an aqueous solution. Direct production as an aqueous solution is therefore advantageous, which is generally accomplished through the reaction of a precious metal precursor with oxalic acid, one of its hydrates or salts in water.

The precious metal precursor may, in particular, be selected from the group consisting of hexahydroxo platinum (IV) acid, its salts, precious metal oxide hydrate, precious metal nitrate, precious metal acetate, and mixtures thereof. Hexahydroxo platinum (IV) acid is particularly suitable as the precious metal precursor, but also its alkali metal salts such as the potassium and sodium salts, K₂Pt(OH)₆ or Na₂Pt(OH)₆. The precious metal precursor may advantageously be added in the form of a solution or suspension, wherein concentrations of 5 wt % to 25 wt %—particularly, 7 wt % 15 wt %—relative to the amount of platinum in water are well suited.

In particular, oxalic acid, oxalic acid dihydrate, sodium oxalate, ammonium oxalate, potassium oxalate, or mixtures thereof may be used as the reaction partner of the precious metal precursor, wherein good results were achieved with oxalic acid and oxalic acid dihydrate.

The amount of oxalic acid, its hydrates or salts used is governed by the amount of platinum in the precious metal precursor. Amounts of approximately 1.7 to 3.1 molar equivalents or 1.8 to 2.8 molar equivalents or 2 to 3 molar equivalents relative to platinum in the precious metal precursor have proven valuable.

The reaction can be started by adding bis(oxalato) platinum (II) acid to the educts, since it has been found that the presence of the end product eases the production of the end product. For this purpose, an amount of 1×10⁻⁴ to 5×10⁻² molar equivalents of precious metal relative to the precious metal in the precious metal precursor may be added. Concentrations of 5×10⁻⁴ to 1×10⁻² or 5×10⁻⁴ to 7×10⁻³ molar equivalents of precious metal relative to the precious metal in the precious metal precursor have likewise proven valuable. The addition may be made as an aqueous solution, wherein concentrations of 5 wt % to 20 wt % or 8 wt % to 15 wt % are usual.

The reaction of the precious metal precursor—particularly, the hexahydroxo platinum (IV) acid with oxalic acid or oxalic acid dihydrate—takes place at temperatures of 0° C. to 20° C., 5° C. to 60° C., 30° C. to 56° C., or 35° C. to 52° C., or at 45° C. to 55° C.

The reaction may be advantageously carried out in this manner at temperatures below the decomposition temperature of the bis(oxalato) platinum (II) acid. In doing so, the difference between reaction temperature and decomposition temperature may be as small as 1° C.—in particular, 5° C. The decomposition temperature is defined as the temperature at the beginning of decomposition, wherein the beginning of decomposition is specified by means of long-term differential thermal analysis in glass ampules at a heating rate of 0.05 K/min according to differential thermal analysis per DIN 51007.

The sequence of additions of educts, precious metal precursor, bis(oxalato) platinum (II) acid, if necessary, and oxalic acid or its salts or hydrates, generally makes no difference. The addition of the educts should be carried out at a temperature below the reaction temperature. Temperatures of, in general, 20° C. to 35° C., or 25° C. to 30° C. have proven valuable. The heating rates may be determined by the onset of the reaction.

The reaction mixture is stirred during the reaction, or, advantageously, even before the addition of the second reaction partner. The addition of the reaction partner is carried out in such a way that the force of the stirrer is sufficient for the basic mixing of the reaction mixture, wherein additional parameters such as stirring conditions, torque of the stirring motor, shape and stability of the stirrer used, concentration (and therefore also the viscosity) of the solution or suspension, as well as the reactor dimensions, play a role.

The development of carbon dioxide and the temperature increase of the exothermic reaction taking place during the reaction may generally allow an inference about the progress and course of the reaction to be drawn, and may be applied as the basis for controlling the addition of oxalic acid, its hydrates, or its salts.

The addition of the oxalic acid, one of its hydrates, or oxalic acid salt can be accomplished continuously or in portions.

The addition of oxalic acid, one of its hydrates, or oxalic acid salt may also be accomplished in several portions in order to better control the reaction, wherein the size of the portions may be the same or different. In this case, it has proven valuable to first add a larger amount, and then complete the reaction with the addition of smaller or ever-decreasing portions.

For example, 0.5 to 1.6 or 0.4 to 1.4 molar equivalents relative to platinum in the platinum precursor (such as platinum(IV) hydroxo acid, in particular) may be added, and the remaining oxalic acid or the remaining oxalic acid salt may be added subsequently, e.g., in several equal amounts. This may therefore happen in one single addition of 0.4 to 1.6 molar equivalents, in 2 further additions of, for instance, 0.2 to 0.8 molar equivalents, or in 3 further additions of, for instance, 0.1 to 0.7 molar equivalents, and so forth.

To produce bis(oxalato) platinum (II) acid as an aqueous solution, in a first step, an aqueous solution or suspension containing at least precious metal precursor, such as hexahydroxo platinum (IV) acid and, if necessary, bis(oxalato) platinum (II) acid is produced,

in a second step, the aqueous solution or suspension from the first step is heated to reaction temperature (i.e., as described above, from approximately 5° C. to approximately 60° C.), and

in a third step, oxalic acid, one of its hydrates, or oxalic acid salt is added.

In a specific embodiment of the method for preparing the aqueous solution of bis(oxalato) platinum (II) acid,
in a first step, a suspension of hexahydroxo platinum (IV) acid in water is prepared;
if necessary, in a second step, a solution of bis(oxalato) platinum (II) acid in water is prepared;
if necessary, the suspension from the first and second steps are combined, and the resulting mixture is heated to the reaction temperature;
in a third step, a first portion of 0.5 to 1.6—particularly, 0.4 to 1.4—molar equivalents of oxalic acid, relative to platinum in platinum(IV) hydroxo acid, is added;
in a fourth step, a second portion of 0.3 to 1.5 or 0.4 to 1.4 molar equivalents of oxalic acid, relative to platinum in platinum(IV) hydroxo acid, is added; and
in a sixth step, the fourth step is optionally repeated one to several times until a total amount of 1.7 to 3.1, or 1.8 to 2.8, or 2 to 3 molar equivalents of oxalic acid relative to platinum in (platinum(IV) hydroxo acid) has been added.

By cooling the aqueous solution of bis(oxalato) platinum (II) acid to a temperature below its freezing point until complete solidification, a shaped object of ice, i.e., frozen water, containing bis(oxalato) platinum (II) acid—which is likewise the subject matter of the present invention—is obtained. Suitable temperatures are generally around −200° C. to 0° C.—particularly, −78.5° C. to −10° C. or −40° C. to −20° C.

The aqueous solution of bis(oxalato) platinum (II) acid may generally contain 1 wt % to 40 wt %, 2 wt % to 27 wt %, or 5 wt % to 19 wt %—in particular, approximately 10 wt %—for instance, 8 wt % to 11 wt %—of bis(oxalato) platinum (II) acid. Higher concentrations lead to a blue coloring, while lower concentrations show a yellow coloring (see Krogmann, Dodel, Chem. Ber. 99 (1966) 3408-3418).

The shaped object may generally have any suitable shape and may, for instance, have an approximately spherical, cuboid, cylindrical, conical, truncated pyramidal, angular, or prismatic shape, or combinations thereof.

To produce it by means of a very simple method, an aqueous solution of bis(oxalato) platinum (II) acid may be poured into a mold and cooled to a temperature below its freezing point until complete solidification. The usual molds for ice cubes, e.g., truncated pyramid forms or ice cube bags, may be used as molds.

The shaped object has the same coloration and concentration of bis(oxalato) platinum (II) acid as does the aqueous solution used as the starting product.

To produce shaped objects of ice containing bis(oxalato) platinum (II) acid, a specific method, in particular, can also be used together with a suitable device, which is capable of producing shaped objects having an approximately spherical shape.

To produce approximately spherical particles, an aqueous solution of bis(oxalato) platinum (II) acid is guided through at least one opening to form droplets, wherein, to form the approximately spherical particles, the droplets are guided to a cooling medium for solidification, the temperature of which is below the freezing point of the aqueous solution of bis(oxalato) platinum (II) acid. The opening may also be configured as a nozzle.

To produce droplets, the liquid jet of the aqueous solution of bis(oxalato) platinum (II) acid may be exposed to vibrations and guided through at least one opening, which may also be configured as a nozzle. The corresponding device comprises, in this case, suitable means for creating the vibrations.

To produce the approximately spherical particles, the droplets at the opening or nozzle are guided to a cooling medium for solidification. The opening or nozzle may thus be immersed in the cooling medium, wherein the temperature of the cooling medium is below the freezing point of the aqueous solution of bis(oxalato) platinum (II) acid.

An advantage may be gained if the opening or nozzle is immersed in the cooling medium, and consists in the fact that the droplets at the outlet opening are guided directly into the cooling medium. Thus, no reduction in cross-section can occur in the outlet opening. The droplets formed by passing through the opening or nozzle thus have a consistent and reproducible size. Since the droplets in this embodiment are introduced into the cooling medium immediately after exiting, no distortions of the droplets occur, which can happen when droplets impact the surface of a cooling medium.

It is, furthermore, advantageous if the temperature of the cooling medium—especially, in the region of the outlet opening of the opening or nozzle—is significantly lower than the freezing point of the aqueous solution of bis(oxalato) platinum (II) acid.

As a result, the thermal load on the cooling medium is correspondingly small. In general, the temperature of the cooling medium in the region of the outlet opening of the opening or nozzle may be between 10° C. and 100° C.—in particular, within a range of 20° C. to 70° C.—below the freezing point of the aqueous solution of bis(oxalato) platinum (II) acid.

In one specific embodiment, the temperature of the cooling medium decreases continuously with increasing distance from the outlet opening. When a droplet passes through the cooling medium, it is continuously solidified by progressively lower temperatures. Thus, the solidification of the droplet does not occur abruptly upon exiting the opening or nozzle. This effect is supported by the fact that, based upon a hypothermic effect of the droplets and the enthalpy of solidification released upon their solidification, the solidification of the droplets occurs only after a certain delay period.

Therefore, when passing through the cooling medium, the droplets may usually first be formed into perfect spheres before they congeal into spherical particles. In this respect, it is especially advantageous that the cooling medium does not have to be moved. The cooling medium may be solid, liquid, or gaseous, and may also change its aggregate state. In the region of the opening or nozzle, the cooling medium is liquid or gaseous. For example, cooled hydrocarbons that are not solid in the required temperature range may be used as the cooling medium, such as n-hexane, n-heptane, or n-octane or their isomers, but also carbon dioxide or nitrogen. For this purpose, liquid nitrogen or even dry ice may be used, which are then present either in solid or liquid form, although they may then also be present in gaseous form in the area of the opening or nozzle by reason of evaporation.

Using this method, spherical particles can be produced from the aqueous solution of bis(oxalato) platinum (II) acid, the shapes of which are formed into perfect spheres and the dimensions of which are subject to only very minor variations. In addition, these spheres have smooth surfaces.

Spherical particles can be produced using the device—particularly, spheres in sizes of mostly 0.1 mm to 1.5 mm in diameter. The spheres thus produced may advantageously be weighed, and thus used in doping the aqueous solution of bis(oxalato) platinum (II) acid without having to re-melt them. In this way, storage life is increased, and the danger of impurities and the loss of precious metals through splashing of the solution avoided.

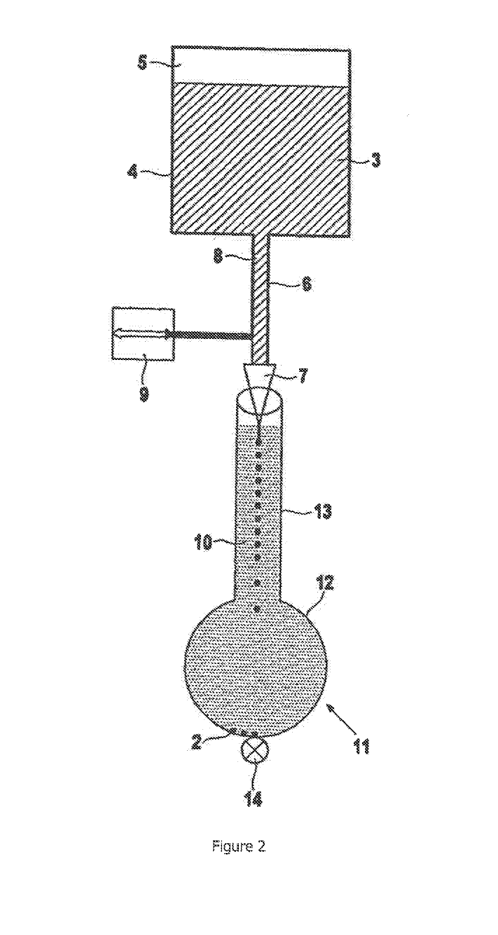

The device is described on the basis of FIG. 2. FIG. 2 shows a schematic representation of a suitable apparatus 1 for producing spherical particles 2 from an aqueous solution of bis(oxalato) platinum (II) acid 3.

The aqueous solution of bis(oxalato) platinum (II) acid 3 is contained in a vessel 4, in the interior of which a protective gas atmosphere 5 with regulable pressure can prevail. The aqueous solution of bis(oxalato) platinum (II) acid 3 in the vessel 4 can, by means of a heating device (not shown), be warmed or cooled to a temperature above the freezing point of the aqueous solution of bis(oxalato) platinum (II) acid. The walls of the vessel 4 are formed to be gas-tight.

A tubular outlet 6 opens up on the underside of the vessel 4 and is guided to an opening or nozzle 7.

A jet of liquid 8 is guided out of the vessel 4 via the outlet 6 to the opening or nozzle 7.

In the present exemplary embodiment, the outlet 6 opens at the bottom of the vessel 4 and runs in a vertical direction, so that the jet of liquid 8 is guided to the opening or nozzle 7 by force of gravity.

Alternatively, the outlet 6 can be directed upwards to the opening 7, so that the jet of liquid 8 is guided to the opening or nozzle 7 only by the effect of pressure. A pressure device—in particular, a gas pressure device—may be provided for this purpose. The advantage of this arrangement is that the aqueous solution of bis(oxalato) platinum (II) acid 3 can be directed to the opening or nozzle 7 in a controlled manner using the generated pressure. This ensures, in particular, that, in the absence of pressure, no aqueous solution of bis(oxalato) platinum (II) acid 3 exits via the opening 7.

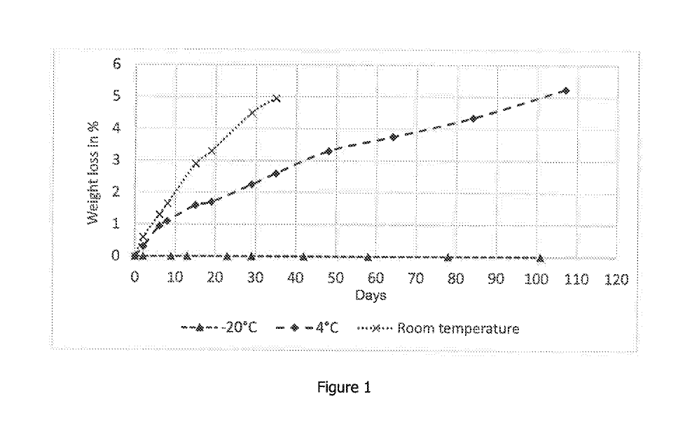

In principle, a pressure device may also be provided in the device 1 according to FIG. 1, in order to there conduct the aqueous solution of bis(oxalato) platinum (II) acid 3 to the opening or nozzle 7 under pressure.

In the present exemplary embodiment, a single opening or nozzle 7 is provided. In principle, multiple arrangements of openings or nozzles 7 may also be provided.

The opening or nozzle 7 and the outlet 6 may likewise be heated, wherein, for this purpose, a separate heating element, likewise not shown, may be provided.

In order that the aqueous solution of bis(oxalato) platinum (II) acid 3 can exit the outlet opening of the opening or nozzle 7 in the form of droplets of defined and constant size, means for the creation of vibrations in the aqueous solution of bis(oxalato) platinum (II) acid 3 may be provided. In principle, this means may be formed by a vibrating plate that is placed in the aqueous solution of bis(oxalato) platinum (II) acid 3 in the interior of the vessel 4.

In FIG. 2, this means is formed of a vibrating element 9, which is coupled to the opening or nozzle 7 either directly or via the outlet 6, in order to transmit these vibrations. The vibrating element 9 may be formed by a piezoelectric element or an electromagnetic vibrating element 9.

The opening or nozzle 7 is immersed in a cooling medium 10, which is stored in a collection vessel 11. The collection vessel 11 has a base body 12, on the upper side of which a hollow, cylindrical column 13 terminates. The column 13 running in the vertical direction is open at its upper side, wherein the opening or nozzle 7 projects into the interior of the column 13 through the open upper side.

For purposes of heating, the collection vessel 11 may be provided with an additional heating device, which can bring it to a desired temperature through heating or cooling. The collection vessel 11 may conveniently be heated so that the temperature of the cooling medium 10 in the column 13 decreases continuously from its upper to its lower side. In principle, a spatially changeable or a constant temperature profile of the cooling medium 10 in the collection vessel 11 may also be selected. In each case, it is ensured that the temperature of the cooling medium 10 at no point in the collection vessel 11 reaches or exceeds the freezing temperature of the aqueous solution of bis(oxalato) platinum (II) acid 3.

The droplets leaving the opening or nozzle 7 congeal upon passing the cooling medium 10 and are deposited on the bottom of the collection vessel 11 as spherical particles 2. A discharge device 14 is located in the region of the bottom of the collection vessel 11, via which the substantially spherical particles 2 can be removed from the collection vessel 11.

In order to attain a continuously operating production process, means are preferably provided for the continuous supply of the aqueous solution of bis(oxalato) platinum (II) acid 3 to the vessel 4. For example, these means may comprise a refilling vessel or the like.

The entire device 1 may also be encapsulated in a vacuum-tight manner, in order to attain a defined protective gas atmosphere 5 within its interior.

The temperature of the jet 8 in the region of the outlet 6 and in the region of the opening or nozzle 7 is preferably maintained at a constant temperature above the freezing point of the aqueous solution of bis(oxalato) platinum (II) acid 3, wherein the temperature is at least 10° C. above the freezing point.

The temperature of the cooling medium 10 in the region of the opening or nozzle 7 is, however, at least 10° C. below the freezing point of the aqueous solution of bis(oxalato) platinum (II) acid.

In general, the temperature of the cooling medium in the region of the opening or nozzle may be between 10° C. and 100° C.—in particular, in a range of 20° C. to 70° C.—below the freezing point of the aqueous solution of bis(oxalato) platinum (II) acid.

The cooling medium may be solid, liquid, or gaseous, and may also change its aggregate state within the device or be present in multiple aggregate states. In the region of the opening or nozzle, the cooling medium is liquid or gaseous. As cooling medium, for example, cooled hexane, heptane, or octane may be used, but also carbon dioxide or nitrogen. For this purpose, liquid nitrogen, in particular, or even dry ice may be used, which are then present either in solid or liquid form, although they may then also be present in gaseous form in the region of the opening or nozzle by reason of evaporation.

In order to produce spherical particles 2 from the aqueous solution of bis(oxalato) platinum (II) acid 3 stored in the vessel 4, the liquid jet 8 in the outlet 6 and, in particular, in the region of the opening or nozzle 7 is caused to vibrate.

According to the principle described by Lord Rayleigh (Proc. Land. Math. Soc. 10.4 (1878)), constrictions are produced in the liquid jet 8 due to the vibrations. Due to these constrictions, the originally homogeneous liquid jet 8 is divided into defined cylindrical sections. With a suitable dimensioning of the cylindrical sections, droplets are formed from the latter upon leaving the opening or nozzle 7, which have a spherical shape when introduced and fed into the cooling medium 10.

In order to produce the desired constrictions in the melt liquid jet 8, the wavelength of the induced vibrations must be greater than the diameter of the melt liquid jet 8. It has been found that the optimal wavelength of the vibrations is about 4.5 times the value of the diameter of the melt liquid jet 8.

The optimal diameter D of the liquid jet 8 for producing spheres with a diameter d is calculated from the condition that the volume of a cylinder segment separated due to the vibrationally induced constrictions in the liquid stream 8 correspond to the volume of these spheres.

The diameter D of this cylinder segment thus corresponds to the diameter of the liquid jet 8. The length L of the cylinder segment corresponds to the vibrationally induced disintegration length L, which corresponds to 4.5 times the value of the diameter D.

Thus, to produce metallic spheres with a diameter d, the optimal value for the diameter D of the liquid jet 8 is given by

D=d/1.89.

In order to generate such a uniform splitting of the liquid jet 8 into sections with a constant disintegration length L, vibrations of constant frequency and amplitude are preferably employed. In this connection, the vibrations may be formed transversely and/or longitudinally with respect to the flow direction of the liquid jet 8.

The disintegration lengths L are essentially predetermined by the spacings of the vibrational nodes in the liquid jet 8.

These spacings are essentially predetermined by the equation L=v T/2, where v denotes the flow velocity of the liquid jet 8 and T denotes the periodicity of the vibration.

Due to the vibrationally induced constriction, droplets of predetermined size are obtained at the opening or nozzle 7. The diameter of the droplets depends upon the diameter of the opening 7, the jet outlet velocity at the opening 7, as well as upon the vibration parameters—in particular, the vibration frequency. These parameters define a process window within which droplets of predetermined and constant size can reproducibly be produced.

Upon exiting, the droplets may, in a particular embodiment, be immersed directly in the cooling medium 10, the temperature of which is below the freezing point. Despite the sudden cooling that occurs, the droplets do not solidify immediately at the opening or nozzle 7, but instead move, still in a liquid state, within the cooling medium 10, as a result of which a perfect spherical shape of the droplets can form.

The rate of the solidification process of the droplets is predeterminable by the temperature of the coolant 10—in particular, the temperature gradient in the longitudinal direction of the column 13. In each case, the collection vessel 11 and, in particular, the height of the column 13, as well as the temperature of the coolant 10, are dimensioned so that the droplets are solidified before they reach the bottom of the collection vessel 11. In this way, undesirable deformations of the spherical particles 2 may be avoided.

In an alternative embodiment, the aqueous solution of bis(oxalato) platinum (II) acid may, in particular, be drained directly from the reactor in which it was produced via an opening—for instance, a valve and, in particular, a bottom drain valve in case it is drained from a reactor—into a flask or a vessel filled with a cooling medium, e.g., liquid nitrogen, and intensely mixed. In this way, also, essentially spherical droplets may be obtained from the frozen, aqueous solution of bis(oxalato) platinum (II) acid. The mixing may be accomplished in different ways, such as by the use of mixers having movable containers, such as a pan mixer, a planetary mixer, or a ring-trough mixer. Likewise, mixers having movable mixing tools such as a propeller stirrer, impeller stirrer, swash-plate stirrer, hollow-blade stirrer, crossbeam stirrer, anchor stirrer, paddle stirrer, grid stirrer, spiral stirrer, or residual quantity stirrer can be used.

The present patent application thus also relates to a method for producing shaped objects of ice containing bis(oxalato) platinum (II) acid, wherein the aqueous solution of bis(oxalato) platinum (II) acid is supplied through at least one opening to a cooling medium, the temperature of which is below the freezing point of the aqueous solution of bis(oxalato) platinum (II) acid and which is stirred to produce particles. In general, the cooling medium is contained in a vessel for this purpose.

The cooling medium may be stirred by a mixer having a movable container, such as a pan mixer, planetary mixer, or ring-trough mixer, or mixers having movable mixing tools such as a propeller stirrer, impeller stirrer, swash-plate stirrer, hollow-blade stirrer, crossbeam stirrer, anchor stirrer, paddle stirrer, grid stirrer, spiral stirrer, residual quantity stirrer, or combinations thereof. The cooling medium may be selected from the cooling media described above and is, advantageously, a liquefied gas—particularly, liquid nitrogen. By means of the above-described method, the product is recovered in the form of a flowable granulate.

In another variant, the shaped objects may be obtained through a method comprising the steps of (1) providing an aqueous solution of bis(oxalato) platinum (II) acid; (2) cooling the aqueous solution of bis(oxalato) platinum (II) acid to a temperature below its freezing point until complete solidification; (3) grinding the frozen aqueous solution of bis(oxalato) platinum (II) acid obtained in the previous step.

The shaped objects thus obtained may, in general, have any suitable form, and may, for example, have an approximately spherical, cuboid, cylindrical, conical, truncated pyramidal, angular, or prismatic shape, or combinations thereof. The size may generally be between 2 μm to 2 cm—in particular, 200 μm to 1 cm or 0.5 mm to 5 mm—depending upon the grinding method or duration.

To conduct step (2), for example, an aqueous solution of bis(oxalato) platinum (II) acid is poured into a mold and cooled to a temperature below its freezing point until complete solidification. The usual molds for ice cubes, e.g., truncated pyramid forms or ice cube bags, may be used as molds.

By means of this method, the storage stability of aqueous solutions of bis(oxalato) platinum (II) acid may successfully be increased, such that, after 35 days—in particular, even after 78 days or 101 days—a loss in weight of less than 2 wt %, less than 1 wt % or 0.5 wt %—in particular, no loss in weight—is observed.

### EXAMPLES

2.75 kg of fully demineralized water was placed into a 10 L reactor having a temperature-controllable jacket and internal thermometer and stirred. 750 g of platinum in the form of approximately 1.875 g of hexahydroxo platinum (IV) acid was transferred to the reactor, and, thereafter, 1.431 kg of oxalic acid dihydrate was added, likewise while stirring, and rinsed with 1 kg of fully demineralized water. Next, the thermocryostat controlling the heating of reactor jacket was turned on, and the contents of the reactor were heated to a temperature of 45° C.

The reaction began with the development of carbon dioxide and formed a blue solution within about 90 minutes. During the reaction, care was taken that the reaction did not exceed 55° C.

After 150 minutes of reaction time, the reaction was tested for complete conversion, and the thermocryostat was set to a temperature of 10° C. Once the internal temperature of the reactor fell to less than 25° C., the thermocryostat was set to a temperature of 20° C., the reactor contents were drained, and the reactor was rinsed with 200 mL of fully demineralized water.

Three samples of 20 g each were removed from the reaction product and weighed in glass flasks with gas-permeable screw tops. One sample was stored at room temperature, one sample at 4° C. in a refrigerator, and the third sample at −20° C. in a freezer. Depending upon the time, the loss in weight of the weighed samples was determined, and the loss in weight in percent plotted against time. The results are shown in FIG. 1.

The uppermost, dotted curve shows the result at room temperature. The individual measurement values are marked with crosses. Over the course of storage, the weight decreased consistently. After 35 days, a loss in weight of 5% was determined, wherein the originally blue solution had become completely colorless and formed a platinum mirror. During storage, the development of a gas could be observed.

The middle, dashed curve shows the result at 4° C. The individual measurement values are marked with diamonds. Compared to the result at room temperature, the sample stored at 4° C. showed a loss in weight of 4.3% at 84 days and a loss in weight of 5.2% after 107 days. After 35 days, the loss in weight was only 2.6%. When the experiment was ended after 107 days, the originally blue solution had become completely colorless and formed a platinum mirror. During storage, the development of a gas could be observed.

The bottom dashed curve shows the result at −20° C. The individual measurement values are marked with triangles. The solution stored at −20° C. congealed, and, even after 78 or 101 days, the weight and coloration remained unchanged. The development of a gas was also not observed.

1. Method for improving the storage stability of aqueous solutions of bis(oxalato) platinum (II) acid, comprising the steps of (1) preparing an aqueous solution of bis(oxalato) platinum (II) acid; (2) cooling the aqueous solution of bis(oxalato) platinum (II) acid to a temperature below its freezing point until complete solidification.

2. Method according to claim 1, wherein the preparation of the aqueous solution of bis(oxalato) platinum (II) acid is accomplished by dissolving bis(oxalato) platinum (II) acid in water or producing bis(oxalato) platinum (II) acid as an aqueous solution.

3. Method according to claim 1, wherein the preparation of the aqueous solution of bis(oxalato) platinum (II) acid is accomplished through the reaction of a precious metal precursor with oxalic acid, one of its hydrates or salts in water.

4. Method according to claim 3, wherein the precious metal precursor is selected from the group consisting of hexahydroxo platinum (IV) acid, its salts, precious metal oxide hydrate, precious metal nitrate, precious metal acetate, and mixtures thereof.

5. Method according to claim 2, wherein, to produce bis(oxalato) platinum (II) acid as an aqueous solution, bis(oxalato) platinum (II) acid is added to the reaction mixture.

6. Method according to claim 2, wherein, to produce bis(oxalato) platinum (II) acid as an aqueous solution, in a first step, an aqueous solution or suspension containing at least precious metal precursor and, if necessary, bis(oxalato) platinum (II) acid is produced,
in a second step, the aqueous solution or suspension from the first step is heated to reaction temperature, and, in a third step, oxalic acid, one of its hydrates, or oxalic acid salt is added.

7. Method according to claim 3, wherein, in a first step, a suspension of hexahydroxo platinum (IV) acid in water is prepared; if necessary, in a second step, a solution of bis(oxalato) platinum (II) acid in water is prepared; if necessary, the suspension from the first and second steps is combined, and the resulting mixture is heated to the reaction temperature; in a third step, a first portion of 0.5 to 1.6—particularly, 0.4 to 1.4—molar equivalents of oxalic acid relative to platinum in platinum(IV) hydroxo acid is added; and, in a fourth step, a second portion of 0.3 to 1.5 or 0.1 to 1.4 molar equivalents of oxalic acid relative to platinum in platinum(IV) hydroxo acid is added; and, in a fifth step, step four is optionally repeated one to several times until a total amount of 1.7 to 3.1, or 1.8 to 2.8, or of 2 to 3 molar equivalents of oxalic acid relative to platinum in platinum(IV) hydroxo acid is added.

8. Shaped object of ice, containing bis(oxalato) platinum (II) acid.

9. Shaped object according to claim 8, which comprises an approximately spherical, cuboid, cylindrical, conical, truncated pyramidal, angular, or prismatic shape, or combinations thereof.

10. Shaped object according to claim 8, which contains 1 wt % to 40 wt %—in particular, 19 wt % to 27 wt %—of bis(oxalato) platinum (II) acid.

11. Method for preparing the shaped object according to claim 8, wherein an aqueous solution of bis(oxalato) platinum (II) acid is poured into a mold and cooled to a temperature below its freezing point until complete solidification.

12. Method for preparing the shaped object according to claim 8, wherein, to produce approximately spherical particles, an aqueous solution of bis(oxalato) platinum (II) acid is guided through at least one opening to produce droplets, wherein, to produce the approximately spherical particles, the droplets are guided for solidification to a cooling medium at the outlet of the opening, the temperature of which is below the freezing point of the aqueous solution of bis(oxalato) platinum (II) acid.

13. Method according to claim 12, wherein the liquid jet exiting the opening is exposed to vibrations.

14. Device for conducting the method according to claim 12, having means for producing droplets from an aqueous solution of bis(oxalato) platinum (II) acid, which comprises means for producing vibrations within a liquid jet and at least one opening—in particular, a nozzle—through which the liquid jet is guided, wherein, to produce spherical particles of an aqueous solution of bis(oxalato) platinum (II) acid, the droplets at the exit of the opening or nozzle are guided to a cooling medium, wherein the opening or nozzle (7) is, if necessary, immersed in the cooling medium (10), and the temperature of the cooling medium (10) is below the freezing point of the aqueous solution of bis(oxalato) platinum (II) acid (3).

15. Method for producing the shaped object according to claim 8, wherein the aqueous solution of bis(oxalato) platinum (II) acid is guided through at least one opening to a cooling medium, the temperature of which is below the freezing point of the aqueous solution of bis(oxalato) platinum (II) acid and which is stirred to produce particles.

16. Method for producing the shaped object according to claim 8, comprising the steps of (1) providing an aqueous solution of bis(oxalato) platinum (II) acid; (2) cooling the aqueous solution of bis(oxalato) platinum (II) acid to a temperature below its freezing point until complete solidification; (3) grinding the frozen aqueous solution of bis(oxalato) platinum (II) acid obtained in the previous step.
